SEQUENCE LISTING (1 sequence)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

(19) United States
(12) Patent Application Publication
(10) Pub. No.: US 2022/0008518 A1
(43) Pub. Date: Jan. 13, 2022

(54) THERAPEUTIC APPROACHES FOR TISSUE RECONSTRUCTION AND WOUND HEALING TREATMENT

(71) Applicant: The Schepens Eye Research Institute, Inc., Boston, MA (US)

(72) Inventors: Yang Liu, Winchester, MA (US); Di Chen, Boston, MA (US); David A. Sullivan, Boston, MA (US)

(21) Appl. No.: 17/292,373

(22) PCT Filed: Nov. 7, 2019
(86) PCT No.: PCT/US19/60286

§ 371 Date: May 7, 2021

Related U.S. Application Data
(60) Provisional application No. 62/757,210, filed on Nov. 8, 2018.

(51) Int. Cl. A61K 38/39; A61K 35/50; A61P 41/00; A61P 17/02

(52) U.S. Cl. CPC A61K 38/39; A61P 17/02; A61P 41/00; A61K 35/50

(57) ABSTRACT

Disclosed are compositions containing a therapeutically effective amount of free recombinant or synthetic lubricin, a lubricin-supplemented preserved AM, or a lubricin-supplemented non-AM substrate for promoting tissue reconstruction and wound healing in a subject and the methods of using thereof. Lubricin-supplemented preserved amniotic membranes and lubricin-supplemented non-AM substrates, and methods of preparing thereof are also described.

### CROSS-REFERENCE TO RELATED APPLICATIONS

This application claims the benefit of priority under 35 U.S.C. § 119(e) to U.S. Provisional Application No. 62/757,210, filed on Nov. 8, 2018, which is incorporated herein by reference in its entirety.

### INCORPORATION BY REFERENCE OF SEQUENCE LISTING

The contents of the sequence listing text file named “36770_580001WO_Sequence_Listing_ST25.txt”, which was created on Nov. 7, 2019 and is 16,384 bytes in size, is hereby incorporated by reference in its entirety.

### FIELD OF THE INVENTION

The present invention relates to treatment for tissue reconstruction and wound healing.

### BACKGROUND

Tissue reconstruction and wound healing are associated with several conditions in various tissues and organs. For example, tissue reconstruction and wound healing are involved in fallopian tube abnormalities, intrauterine adhesions, and associated female infertility. Other exemplary conditions associated with tissue reconstruction and wound healing include ocular surface diseases and dacryocystitis. Chronic tear retention and stasis induced by dacryocystitis can lead to secondary infection. Hence, there exists a need for safe and effective treatments for tissue reconstruction and wound healing.

### SUMMARY

The disclosure provides compounds and methods for promoting tissue repair or healing, e.g., treating tissue reconstruction and/or wound healing, in a subject with free lubricin, a lubricin-supplemented preserved amniotic membrane (AM), or a lubricin-supplemented non-AM substrate. For example, purified lubricin comprises residues 25-1404 of SEQ ID NO:1 or residues 1-1404 of SEQ ID NO:1 (residues 1-24 representing a signal sequences. Fragments of lubricin with wound-healing activity are also within the invention.

Also, included are lubricin-supplemented preserved amniotic membranes and lubricin-supplemented non-AM substrates, and methods of preparing thereof. In some examples, the compositions and methods encompass a substrate that does not include AM, e.g., mammalian derived amniotic tissue.

An aspect of the invention includes compositions useful for treating tissue reconstruction and wound healing in a subject. For example, a composition for treating tissue reconstruction and wound healing in a subject contains a therapeutically effective amount of free recombinant or synthetic lubricin, a lubricin-supplemented preserved amniotic membrane (AM) or a lubricin-supplemented non-AM substrate. The preserved AM of the lubricin-supplemented preserved AM may be a cryopreserved AM (CP-AM) or a freeze dried AM (FD-AM). Furthermore, the lubricin-supplemented preserved AM is a FD-AM that has been soaked in, rehydrated with, or incubated with lubricin for at least 1, 3, 6, 12, 24, 36, 48 hours or more. For example, the FD-AM has been incubated with lubricin overnight. Similarly, the lubricin-supplemented non-AM substrate is a non-AM substrate that has been soaked in, rehydrated with, or incubated with lubricin for at least 1, 3, 6, 12, 24, 36, 48 hours or more. For example, the non-AM substrate has been incubated with lubricin overnight.

The compositions contain a therapeutically effective amount of free recombinant or synthetic lubricin, a lubricin-supplemented preserved AM or a lubricin-supplemented non-AM substrate with means to promote tissue reconstruction, prevent adhesion formation, alleviate obstructions, facilitate wound healing or perform any combinations thereof. The composition promotes tissue reconstruction of an ocular surface, an oral surface, a periodontal surface, an abdominal surface, a vaginal surface, a cervical surface, a uterine surface, a skin surface or a mucosal surface. The composition also prevents adhesion formation of an ocular surface, nasolacrimal duct, intrauterine, fallopian tube, or post-radiation tissue damage. The composition further alleviates obstructions of nasolacrimal duct or fallopian tube, or facilitates wound healing of a burn injury, an epithelial defect or an ulcer.

For example, the composition containing lubricin alone, e.g., free recombinant or synthetic lubricin, i.e., purified lubricin that is not bound to or associated with AM or other membrane, is used for treating inflammation or inflamed tissue(s). For such applications, lubricin may be the sole active ingredient, i.e., the formulation may contain other ingredients such as inactive compounds (carrier or expients). More specifically, the composition containing free recombinant or synthetic lubricin is used for treating fallopian tube inflammation or inflammation of other bodily lumens, ducts, or tubular structures in the body. Other exemplary lumens/tubular include nasolacrimal ducts. In some cases, inflammation of such tissues leads to obstruction or closing up of the inflamed lumen, which consequently leads to loss of function of the tissue. The composition containing free recombinant or synthetic lubricin (i.e., lubricin not bound to AM or another membrane) is useful for treating obstructions. Lubricin this type of formulation can be a solution, e.g., an aqueous solution or solution containing non-aqueous carriers or excipients, is used to contact the lumen tissue, e.g., directly contact the tissue of the lumen or tubular structure, e.g., by lavage or infusion of the lumen or struction. The lubricin solution/composition binds to the tissue that comprises an obstruction and leads to re-opening of the cavity/lumen. The treatment leads to removal of the obstruction and restoration of the function of the lumen, duct, tubular structure, or cavity of the bodily tissue.

The composition may be used to facilitate wound healing or tissue reconstruction of an ocular surface disease. For example, the composition may be used to relieve or reduce a sign of conjunctival congestion, conjunctival/cornea ulceration, corneal edema, corneal clouding, extensive fluorescence staining, neovascularization, aqueous flare, aqueous cells in anterior chamber, conical perforation, corneal/conjunctival epithelial demarcation, conical stromal inflammation, conical thinning, cornea stromal edema, corneal endothelial inflammatory plaque, Descemet's folds, conjunctival mucopurulent discharge, anterior chamber reaction and hypopyon, upper eyelid edema, posterior synechiae, hyphema, high intraocular pressure, loss of ocular contents, iris prolapse, or severe dry eye. The composition may also be used to reduce or relieve a symptom or a sign of dacryocystitis.

The lubricin-supplemented preserved AM or the lubricin-supplemented non-AM substrate may be sutured to the surface. The lubricin-supplemented preserved AM or the lubricin-supplemented non-AM substrate may come off after a certain time, e.g., a week, two weeks or three weeks. Alternatively, the lubricin-supplemented preserved AM or the lubricin-supplemented non-AM substrate may be dissolved and/or degraded. Or, the lubricin-supplemented preserved AM or the lubricin-supplemented non-AM substrate may need to be removed as necessary and/or periodically replaced. An exemplary drug-delivery form of the compositions containing a therapeutically effective amount of free recombinant or synthetic lubricin, a lubricin-supplemented preserved AM or a lubricin-supplemented non-AM substrate is a form of contact lens.

The invention also encompasses methods of treating tissue reconstruction and wound healing in a subject. For example, a method of treating tissue reconstruction and wound healing in a subject includes the step of administering a composition containing a therapeutically effective amount of free recombinant or synthetic lubricin, a lubricin-supplemented preserved AM, or a lubricin-supplemented non-AM substrate to the subject. Preferably, the tissue or wound is directly contacted with free recombinant or synthetic lubricin, a lubricin-supplemented preserved AM, or a lubricin-supplemented non-AM substrate. For another example, the method uses a lubricin-supplemented preserved AM, wherein the preserved AM is a cryopreserved AM or a freeze dried AM. The lubricin-supplemented preserved AM is preferably FD-AM that has been soaked in, rehydrated with or incubated with lubricin at least 1, 3, 6, 12, 24, 36, 48 hours or more. For instance, the FD-AM has been incubated with lubricin overnight. For additional example, the method uses a lubricin-supplemented non-AM substrate. The lubricin-supplemented non-AM substrate is preferably a non-AM substrate that has been soaked in, rehydrated with or incubated with lubricin at least 1, 3, 6, 12, 24, 36, 48 hours or more. For instance, the non-AM substrate has been incubated with lubricin overnight.

Based on the effects of the compositions described above, the methods are designed to promote tissue reconstruction, prevent adhesion formation, alleviate obstructions, facilitate wound healing or perform any combinations thereof. The methods may be employed to promote tissue reconstruction of an ocular surface, an oral surface, a periodontal surface, an abdominal surface, a vaginal surface, a cervical surface, a uterine surface, a skin surface or a mucosal surface; prevent adhesion formation of an ocular surface, nasolacrimal duct, intrauterine, fallopian tube, or post-radiation tissue damage; alleviate obstructions of nasolacrimal duct or fallopian tube; or facilitate wound healing of a burn injury, an epithelial defect or an ulcer. For example, the methods are employed to treat inflammation by using the composition containing free recombinant or synthetic lubricin, i.e., lubricin that is not bound to or associated with a membrane. More specifically, the methods are employed to treat fallopian inflammation or nasolacrimal duct obstruction by using the composition containing free recombinant or synthetic lubricin. For another example, the methods are employed to alleviate obstructions, as free recombinant or synthetic lubricin binds to the tissue with obstruction and re-opens the cavity, consequently removing obstruction.

Accordingly, the methods of treating tissue reconstruction and wound healing in a subject may be used to reduce or relieve a symptom or a sign of a fallopian tube abnormality, an intrauterine adhesion or associated female infertility. Exemplary symptoms or signs may be associated with a pelvic inflammatory disease, a pathogen infection, endometriosis, an adhesion from previous surgery, an adhesion from nontubal infection, pelvic tuberculosis, salpingitis isthmica nodosa, a plug of mucus and amorphous debris, a spasm of a uterotubal ostium, a hydrosalpinx or any combinations thereof. Exemplary signs of fallopian tube obstruction include infertility, changes in a laparoscopy or a hysterosalpingogram, which shows a blockage or if the dye flows freely into the abdomen.

Alternatively, the methods may be used to reduce or relieve a symptom or a sign of ocular surface diseases or dacryocystitis. Exemplary signs of ocular surface diseases that require wound healing and/or tissue reconstruction include conjunctival congestion, conjunctival/cornea ulceration, corneal edema, conical clouding, extensive fluorescence staining, neovascularization, aqueous flare, aqueous cells in anterior chamber, corneal perforation, corneal/conjunctival epithelial demarcation, corneal stromal inflammation, corneal thinning, cornea stromal edema, conical endothelial inflammatory plaque, Descemet's folds, conjunctival mucopurulent discharge, anterior chamber reaction and hypopyon, upper eyelid edema, posterior synechiae, hyphema, high intraocular pressure, loss of ocular contents, iris prolapse, and severe dry eye.

Another aspect of the invention is lubricin-supplemented preserved AMs. For example, the preserved AM is a CP-AM or a FD-AM. Alternatively, the preserved AM is a FD-AM, more specifically, a FD-AM that has been soaked in, rehydrated with or incubated with lubricin at least 1, 3, 6, 12, 24, 36, 48 hours or more. For example, the FD-AM has been incubated with lubricin overnight.

The invention also includes is lubricin-supplemented non-AM substrates. The substrate (membrane, carrier or template) is composed of materials suitable to be used instead of AM (e.g., polymers, hydrogel) with suitable characteristics, including flexibility, thickness, transparency, etc. For instance, the substrate is suitable to be used on the eyes. For example, the materials are hydrogels based upon polymethyl methacrylate (PMMA) or silicone, which are already used as contact lens materials. For another example, the substrate has high modulus similar to contact lens, as shown in the following table (see, also, Snyder, Contact Lens Spectrum, 2007 February, which is hereby incorporated by reference in its entirety):

Modulus Values and Parameters of Silicone
Hydrogel and Hydrogel Lenses

| | Modulus | BASE CURVE | DIAMETER |
|---|---|---|---|
| Lens | (MPA) | (MM) | (MM) |
| Night & Day | 1.4¹ | 8.4/8/6 | 13.8 |
| (CIBA) | | | |
| O₂Optix | 1.2¹ | 8.6 | 14.2 |
| (CIBA) | | | |
| PureVision | 1.1¹ | 8.6 | 14.0 |
| (B&L) | | | |
| Acuvue Oasys | 0.7² | 8.4 | 14.0 |
| (Vistakon) | | | |
| Acuvue Advance | 0.4¹ | 8.3/8.7 | 14.0 |
| (Vistakon) | | | |

¹Ross et al. Silicone Hydrogels: Trends in Products and Properties. Presented at BCLA 29th Clinical Conference & Exhibition, Brighton, UK; 3-5 Jun., 2005.
²Manufacturer's value.

For further example, the substrate has sufficient transparency to be used on the eyes. In general, the term “transparency” is characterized by a material's Transmittance. Transmittance is a dimensionless parameter (or given as a percentage) of the ratio of transmitted light intensity to incident light intensity. An American Society for Testing and Materials (ASTM) standard regarding Transmittance, which discloses the definitions and measurement techniques of transparency, is attached herein as an APPENDIX and hereby incorporated by reference in its entirety. The ASTM standard is applicable to any translucent or transparent material. Measurement of Transmittance in connection with materials to be used on the eyes, e.g., silicone hydrogel contact lenses, has been known in the art (see, e.g., Fuentes, et al. Proc. SPIE 8785, 8785AZ (18 Nov. 2013); doi: 10.1117/12.2025710; https://doi.org/10.1117/12.2025710; and Alyanak, Int. J. Artif Organs. 1991, 14(2):116-121, all of which are hereby incorporated by reference in their entireties). Examplary materials with suitable transparency to be used on the eyes, e.g., for contact lenses, include polymethyl methacrylate (PMMA: Acrylic), polycarbonate (PC), polystyrene (PS), polyvinylchloride (PVC), polyesters (PET, PBT), and polyamide (PA: Naylon) (see, e.g., Findik, ISRN Mechanical Engineering, Volume 2011, Article ID 160671, 4 pages, which is hereby incorporated by reference in its entirety).

Other exemplary non-AM substrates include photo-crosslinkable sericin hydrogel, decellularized animal skin, decellularized animal cornea, and chitosan, cellulose, collagen and gelatin, hyaluronic acid, poly(lactide-co-glycolide), polyurethanes, poly(ethylene glycol), polycaprolactone (see, e.g., Qi et al., Biomater Sci. 2018; 6:2859-70; Kuna et al., Cell Transplant. 2017; 26:293-307; Choi et al., Xenotransplantation. 2018:e12446; and Savoji et al., Front Bioeng Biotechnol. 2018; 6:86, all of which are hereby incorporated by reference in their entireties). Exemplary non-AM substrates to be used on skin are described in, for example, Senthil et al., Int J Artif Organs. 2018 August; 41(8):467-473; Tarusha et al., J Mater Sci Mater Med. 2018 Feb. 2; 29(3):22; Takei et al., J Biosci Bioeng. 2018 April; 125(4):490-495; Baghaie et al., J Biomater Sci Polym Ed. 2017 December; 28(18):2220-2241; Kaygusuz et al., Int J Biol Macromol. 2017 December; 105(Pt 1):1161-1165; Poonguzhali et al., Int J Biol Macromol. 2017 December; 105(Pt 1):111-120; Song et al., Mater Sci Eng C Mater Biol Appl. 2017 Oct. 1; 79:866-874; and Ampawong et al., J Biomater Sci Polym Ed. 2017 September; 28(13):1286-1302, and exemplary non-AM substrates to be used on cornea are described in, for example, Hashemi et al., Indian J Ophthalmol. 2018 February; 66(2):225-228 and Gallagher et al., Adv Healthc Mater. 2016 August; 5(16):2013-8, all of which are hereby incorporated by reference in their entireties.

The lubricin-supplemented non-AM substrate is a non-AM substrate that has been soaked in, rehydrated with or incubated with lubricin at least 1, 3, 6, 12, 24, 36, 48 hours or more. For example, the non-AM substrate has been incubated with lubricin overnight.

The invention also provides methods of preparing a lubricin-supplemented preserved AM or non-AM substrate. For example, the method of preparing a lubricin-supplemented preserved AM or a lubricin-supplemented non-AM substrate includes the step of incubating a preserved AM or non-AM substrate with lubricin for sufficient time to rehydrate and/or supplement the membrane with lubricin. The preserved AM to be used for these methods may be a CP-AM or a PD-AM. More particularly, the preserved AM to be used for these methods may be a FD-AM.

The invention further provides pharmaceutical compositions to be used for treating tissue reconstruction and wound healing in a subject. The pharmaceutical compositions may contain a therapeutically effective amount of free recombinant or synthetic lubricin, a lubricin-supplemented preserved AM, or a lubricin-supplemented non-AM substrate, and a pharmaceutically acceptable carrier and/or excipient. An excipient or carrier is an inactive substance or comprises inactive substances that serves/serve as the vehicle or medium for a drug or other active substance. Exemplary pharmaceutically acceptable carriers include a compound selected from the group consisting of a physiological acceptable salt, poloxamer analogs with carbopol, carbopol/hydroxypropyl methyl cellulose (HPMC), carbopol-methyl cellulose, carboxymethylcellulose (CMC), hyaluronic acid, cyclodextrin, and petroleum. The physiological acceptable salt may be an ophthalmically acceptable balanced salt solution. Exemplary ophthalmically acceptable balanced salt solutions include a one or more electrolytes selected from the group consisting of sodium phosphate, sodium chloride, potassium chloride, sodium bicarbonate, potassium bicarbonate, calcium chloride, magnesium chloride, trisodium citrate, hydrochloric acid, and sodium bicarbonate. The ophthalmic composition may also comprise one or more ophthalmically acceptable agents.

The compositions and methods described herein are useful for a subject, wherein the subject is a mammal in need of such treatment, e.g., a subject that has been diagnosed with or showed symptoms or signs of tissue damage due to medical intervention, e.g., surgery, or due to experiencing a wound. The mammal is, e.g., a human, a primate, a mouse, a rat, a dog, a cat, a horse, as well as livestock or animals grown for food consumption, e.g., cattle, sheep, pigs, chickens, and goats. Preferably, the mammal is a human.

The compositions described herein are administered topically. Preferably, the wound or surgical site is directly contacted with lubricin, lubricin-supplemented AMs or lubricin-supplemented non-AM substrates. In a preferred embodiment, the composition is administered shortly after diagnosis or appearance of a sign or symptom of tissue damage as determined by a medical practioner using standard methods. In some aspects, the composition is administered within 1 minute, 5 minutes, 10 minutes, 15 minutes, 30 minutes, 45 minutes, 1 hour, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, 30 hours, 36 hours, 42, hours, 48 hours, 56 hours or 72 hours after diagnosis or appearance of a sign. Alternatively, the composition is administrated when medically appropriate.

All compounds of the invention are purified and/or isolated. Specifically, as used herein, an “isolated” or “purified” small molecule, nucleic acid molecule, polynucleotide, polypeptide, or protein, is substantially free of other cellular material, or culture medium when produced by recombinant techniques, or chemical precursors or other chemicals when chemically synthesized. A purified polypeptide or protein does not include amino acid sequences that flank a reference sequence (e.g., SEQ ID NO: 1) in its naturally-occurring state. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 96%, 97%, 98%, 99%, or 100% (w/w) of the desired compound by weight. Purity is measured by any appropriate standard method, for example, by column chromatography, thin layer chromatography, or high-performance liquid chromatography (HPLC) analysis. Purified also defines a degree of sterility that is safe for administration to a human subject, e.g., lacking infectious or toxic agents.

Similarly, by “substantially pure” is meant a polypeptide that has been separated from the components that naturally accompany it. Typically, the polypeptides are substantially pure when they are at least 60%, 70%, 80%, 90%, 95%, or even 99%, by weight, free from the proteins and naturally-occurring organic molecules with they are naturally associated.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to achieve a beneficial clinical effect in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms or signs, eliminate the symptoms or signs and/or their underlying cause, and/or facilitate improvement or remediation of damage. The terms “inhibiting” and “inhibition” of a disease in a subject means preventing or reducing the progression and/or complication of condition, disorder, or disease in the subject. For example, inhibition includes inhibiting adhesion formation.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Each embodiment disclosed herein is contemplated as being applicable to each of the other disclosed embodiments.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF THE DRAWINGS

FIGS. 1A-1H are a series of images showing lubricin expression in human AMs and placental tissues:

FIG. 1A shows identification of lubricin protein in human AMs (A) and placentas (P) by Western blot. Results from 4 different samples are shown, as well as the rh-lubricin control.

FIG. 1B shows staining of placental chorionic villus stems (V) by hematoxylin and eosin (H&E).

FIG. 1C shows immunofluorescence staining of lubricin (red) in placental chorionic villi (V), with 4′,6-diamidino-2-phenylindole (DAPI) counterstaining of nuclei (blue).

FIG. 1D shows staining of the human AM by H&E.

FIG. 1E shows immunofluorescence staining of lubricin (red) in human AM epithelial and stromal cells.

FIGS. 1F-1H show CP-AM epithelia (predominantly) (FIG. 1F) and FD-AM before (no staining) (FIG. 1G) and after (FIG. 1H) lubricin exposure, with sections counterstained with DAPI (blue). All AMs were positioned with the epithelial side up. All scale bars equal 25 μM. H&E is an acronym for hematoxylin and eosin; DAPI is an acronym for 4′,6-diamidino-2-phenylindole.

### DETAILED DESCRIPTION

Lubricin is a mucin-like glycoprotein, e.g., as described in Lambiase A et al., Ocul Surf; 2017; 15:77-87; Schmidt T A et al., JAMA Ophthalmol. 2013; 18:1-11; and U.S. Pat. Nos. 8,980,840, 8,680,057, 8,026,346, 7,618,941, 7,001,881 and 6,743,774, all of which are hereby incorporated by reference in their entireties. A representative amino acid sequence of lubricin is as follows (SEQ ID NO: 1; Accession No. Q92954.3 at https://www.ncbi.nlm.nih.gov/protein/Q92954.3, which is hereby incorporated by reference in its entirety):

MAWKTLPIYLLLLLSVFVIQQVSSQDLSSCAGRCGEGYSRDATCNCDYNC
QHYMECCPDFKRVCTAELSCKGRCFESFERGRECDCDAQCKKYDKCCPDY
ESFCAEVHNPTSPPSSKKAPPPSGASQTIKSTTKRSPKPPNKKKTKKVIE
SEEITEEHSVSENQESSSSSSSSSSSSTIRKIKSSKNSAANRELQKKLKV
KDNKKNRTKKKPTPKPPVVDEAGSGLDNGDFKVTTPDTSTTQHNKVSTSP
KTTIAKPINPRPSLPPNSDTSKETSLTVNKETTVETKETTTTNKQTSTDG
KEKTTSAKETQSIEKTSAKDLAPTSKVLAKPTPKAETTTKGPALTTPKEP
TPTTPKEPASTTPKEPTPTTIKSAPTTPKEPAPTTTKSAPTTPKEPAPTT
TKEPAPTTPKEPAPTTTKEPAPTTTKSAPTTPKEPAPTTPKKPAPTTPKE
PAPTTPKEPTPTTPKEPAPTTKEPAPTTPKEPAPTAPKKPAPTTPKEPAP
TTPKEPAPTTTKEPSPTTPKEPAPTTTKSAPTTTKEPAPTTTKSAPTTPK
EPSPITTKEPAPTTPKEPAPTTPKKPAPTTPKEPAPTTPKEPAPTTIKKP
APTTPKEPAPTTPKETAPTTPKKLTPTTPEKLAPTTPEKPAPTTPEELAP
TTPEEPTPTTPEEPAPTTPKAAAPNTPKEPAPTTPKEPAPTTPKEPAPTT
PKETAPTTPKGTAPTTLKEPAPTTPKKPAPKELAPTTTKEPTSTTSDKPA
PTTPKGTAPTTPKEPAPTTPKEPAPTTPKGTAPTTLKEPAPTTPKKPAPK
ELAPTTTKGPTSTTSDKPAPTTPKETAPTTPKEPAPTTPKKPAPTTPETP
PPTTSEVSTPTTTKEPTTIHKSPDESTPELSAEPTPKALENSPKEPGVPT
TKTPAATKPEMTTTAKDKTTERDLRTTPETTTAAPKMTKETATTTEKTTE
SKITATTTQVTSTTTQDTTPFKITTLKTTTLAPKVTTTKKTITTTEIMNK
PEETAKPKDRATNSKATTPKPQKPTKAPKKPTSTKKPKTMPRVRKPKTTP
TPRKMTSTMPELNPTSRIAEAMLQTTTRPNQTPNSKLVEVNPKSEDAGGA
EGETPHMLLRPHVFMPEVTPDMDYLPRVPNQGIIINPMLSDETNICNGKP
VDGLTTLRNGTLVAFRGHYFWMLSPFSPPSPARRITEVWGIPSPIDTVFT
RCNCEGKTFFFKDSQYWRFTNDIKDAGYPKPIFKGFGGLTGQIVAALSTA
KYKNWPESVYFFKRGGSIQQYIYKQEPVQKCPGRRPALNYPVYGETTQVR
RRRFERAIGPSQTHTIRIQYSPARLAYQDKGVLHNEVKVSILWRGLPNVV
TSAISLPNIRKPDGYDYYAFSKDQYYNIDVPSRTARAITTRSGQTLSKVW
YNCP

Residues 1-24 of SEQ ID NO:1 represent a signal sequences. Residues 25-1404 represent the mature protein.

The protein or fragment thereof includes glycosylation at one of more of the following sites:

SER-123; SER-136; THR-240; THR-253; THR-277;

THR-291; THR-305; SER-306; THR-310; SER-317; THR-324; THR-332;

THR-338; THR-367; SER-373; THR-376; THR-384; THR-385; SER-388;

THR-391; THR-399; THR-400; THR-407; THR-408; THR-415; THR-423;

SER-427; THR-430; THR-438; THR-439; THR-446; THR-447; THR-454;

THR-455; THR-477; THR-478; THR-485; THR-493; THR-494; THR-501;

THR-502; THR-509; THR-525; SER-529; THR-532; THR-540; THR-541;

SER-553; THR-555; THR-563; THR-564; THR-571; THR-572; THR-579;

THR-580; THR-587; THR-588; THR-595; THR-603; THR-604; THR-611;

THR-612; THR-616; THR-619; THR-627; THR-676; THR-683; THR-684;

THR-691; THR-692; THR-699; THR-700; THR-704; THR-707; THR-723;

THR-724; THR-736; THR-768; THR-769; THR-776; THR-777; THR-792;

THR-793; THR-805; SER-812; THR-829; THR-837; THR-838; SER-892;

THR-900; THR-930; THR-931; SER-962; THR-963; THR-968; THR-975;

THR-978; THR-979; THR-980; THR-1039 AND/OR THR-1161.

Other features of the protein include the following regions, domains, bonds or sites:
The present invention relates to the uses of the glycoprotein lubricin (also known as proteoglycan 4 (PRG4), articular superficial zone protein, megakaryocyte stimulating factor, or tribonectin: see, for example, Schmidt et al., Ophthalmol 2013; 18:1-11; Jay et al., J. Orthop. Res. 2001, 19(4):677-87; and Flannery et al. Biochem Biophys Res Commun. 1999, 254(3):535-41, all of which are hereby incorporated by reference in their entireties). Similar yet unrelated examples of using lubricin and/or its derivatives, e.g., tribonectin, for treating decreased vaginal boundary lubrication or degenerative joint disorders, or lubricating friction between a tissue surface and an artificial device are described in, e.g., U.S. Pat. Nos. 8,980,840, 8,680,057, 8,026,346, 7,618,941, 7,001,881 and 6,743,774, all of which are hereby incorporated by reference in their entireties. In addition to the full-length protein, e.g., SEQ ID NO:1 shown above, the invention encompasses use of fragments of lubricin, provided the fragments comprises wound-healing, tissue reconstruction and/or lubricating activity, e.g., at least 10%, 20%, 50%, 75%, 100% or 2-fold, 5-fold, 10-fold or more of such activity compared to the glycoprotein, lubricin (glycosylated), shown above. A fragment is a portion of the full-length mature protein that is less than the length of the full-length protein. For example, a fragment may be less than 1404 residues, less than 1000 residues, less than 500 residues, less than 250 residues, or less than 100 residues. A fragment of the protein may also encompass internal deletions such as the absence of regions up to 1, 2, 3, 4, 5, 10, 15, 20, 25, 50, 75, 100, 250, or 500 contiguous residues (compared to SEQ ID NO:1). Fragments are useful in the therapeutic methods provided that they are characterized as having wound-healing, tissue reconstruction and/or lubricating activity of the full length lubricin protein as described above.

In particular, the present invention encompasses compositions and methods utilizing lubricin alone as well as using lubricin bound to a preserved amniotic membrane or a non-AM substrate for promoting tissue reconstruction (e.g., ocular surface, oral, periodontal, abdominal, vaginal, cervical and uterine), preventing adhesion formation (e.g., ocular surface, nasolacrimal duct, intrauterine, fallopian tube, post-radiation tissue damage), alleviating obstructions (e.g., nasolacrimal duct, fallopian tube), and facilitating wound healing (e.g., burn injuries, epithelial defects, ulcers).

AM is the inner fetal membrane that encloses the amniotic cavity and fetus, and has two different sides: the epithelial and the stromal side. AM has anti-inflammatory, anti-adhesive, anti-angiogenic and anti-microbial properties, and has been widely used in tissue reconstruction and wound healing, especially on the ocular surface. For practical reasons, human AMs are typically preserved by cryopreservation (cryopreserved amniotic membrane, CP-AM) or by freeze-drying (freeze dried amniotic membrane, FD-AM).

Lubricin is an anti-adhesive and anti-inflammatory boundary lubricant that was first identified in synovial fluid. Lubricin is also produced by human ocular surface epithelial cells, and has been shown to significantly reduce friction and shear stress at the ocular surface. As disclosed herein, freshly grafted human AM and placenta (positive control) have now been found to contain lubricin. Lubricin expression was observed along the chorionic villi in the placenta, and both sides of fresh AM as well as in the epithelial side of CP-AM. However, no lubricin was observed in FD-AM. After incubating with lubricin for a period of time, e.g., overnight, FD-AM showed lubricin immunoreactivity on both sides of the membrane.

This discovery indicates that lubricin serves to provide the lubricating, anti-adhesive and anti-inflammatory properties of the AM. Cryopreservation and freeze-drying processes cause a reduction or loss of lubricin in AM and a decrease or loss of lubricin's function in tissue reconstruction and wound healing. Addition of lubricin to AM, as described herein, restores AM's function for clinical use.

Exemplary embodiments of the invention include using lubricin alone and/or supplementation of lubricin on preserved AM or non-AM substrate as safe and effective treatments for fallopian tube abnormalities, intrauterine adhesions, and associated female infertility. Fallopian tube disease and pelvic adhesions prevent normal transport of the oocyte and sperm through the fallopian tube.

The primary cause of tubal factor infertility is pelvic inflammatory disease caused by pathogens such as chlamydia or gonorrhea. Other conditions that may interfere with tubal transport include severe endometriosis, adhesions from previous surgery or nontubal infection (e.g., appendicitis, inflammatory bowel disease), pelvic tuberculosis, and salpingitis isthmica nodosa (i.e., diverticulosis of the fallopian tube). Proximal tubal blockage may result from plugs of mucus and amorphous debris or spasm of the uterotubal ostium, but does not reflect true anatomic occlusion. Women with distal tubal obstruction may develop hydrosalpinges, which decrease the success rate of in vitro fertilization.

Additional exemplary embodiments of the invention include the use of lubricin alone and/or supplementation of lubricin on preserved AM or non-AM substrate as safe and effective treatments for ocular surface diseases or dacryocystitis. Ocular surface diseases, such as conjunctival congestion, conjunctival/cornea ulceration, corneal edema, conical clouding, extensive fluorescence staining, neovascularization, aqueous flare, aqueous cells in anterior chamber, conical perforation, corneal/conjunctival epithelial demarcation, conical stromal inflammation, conical thinning, cornea stromal edema, corneal endothelial inflammatory plaque, Descemet's folds, conjunctival mucopurulent discharge, anterior chamber reaction and hypopyon, upper eyelid edema, posterior synechiae, hyphema, high intraocular pressure, loss of ocular contents, iris prolapse, and severe dry eye, may involve require wound healing and/or tissue reconstruction using the methods and compositions described herein.

Inflammation of the lacrimal sac (acute dacryocystitis) has various causes, and in most cases the common factor is complete nasolacrimal duct (NLD) obstruction that prevents normal drainage from the lacrimal sac into the nose. Chronic tear retention and stasis lead to secondary infection. Complications include dacryocystocele formation, chronic conjunctivitis, and spread to adjacent structures (orbital or facial cellulitis). Dacryocystitis indicating total NLD obstruction requires a dacryocystorhinostomy in most cases because of inevitable persistent epiphora and recurrent infection. Chronic dacryocystitis, a smoldering low-grade infection, may develop in some individuals. This usually results in distension of the lacrimal sac, and diagnostic probing and irrigation do not achieve permanent patency in adults. Chronic dacryocystitis needs to be surgically resolved before elective intraocular surgery.

Lubricin's presence in the AM accounts for the anti-adhesive, anti-inflammatory and lubricating properties of the AM. The findings described herein indicate therapies for tissue reconstruction and wound healing. Prior to the invention, lubricin and/or lubricin-coated preserved AM had not been used for tissue reconstruction and wound healing and/or promoting tissue reconstruction (e.g., tissue types: ocular surface, oral, periodontal, abdominal, vaginal, cervical and uterine), preventing adhesion formation (e.g., tissue types: ocular surface, nasolacrimal duct, intrauterine, fallopian tube, post-radiation tissue damage), alleviating obstructions (e.g., tissue types: nasolacrimal duct, fallopian tube), or facilitating wound healing (e.g., burn injuries, epithelial defects, ulcers).

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 96%, 97%, 98%, 99%, or 100% (w/w) of the desired compound by weight. Purity is measured by any appropriate standard method, for example, by column chromatography, thin layer chromatography, or high-performance liquid chromatography (HPLC) analysis. Purified also defines a degree of sterility that is safe for administration to a human subject, e.g., lacking infectious or toxic agents.

Similarly, by “substantially pure” is meant a polypeptide that has been separated from the components that naturally accompany it. Typically, the polypeptides are substantially pure when they are at least 60%, 70%, 80%, 90%, 95%, or even 99%, by weight, free from the proteins and naturally-occurring organic molecules with they are naturally associated.

“Recombinant” refers to an artificial combination of two otherwise separated segments of sequence, e.g., by chemical synthesis or by the manipulation of isolated segments of nucleic acids or amino acids by genetic engineering techniques. For example, the recombinant lubricin is produced, as described in EP3060577A1, which is hereby incorporated by reference in its entirety.

Lubricin is a glycosylated protein of which glycosylation sites (e.g., Ser/Thr residues) are glycosylated at the level of 20-100%. For example, at least 20%, 25%, 30%, 35%, 40%, 45%, 50%, 55%, 60%, 65%, 70%, 75%, 80%, 85%, 90%, 95%, or 100% of the glycosylation sites of lubricin are glycosylated. See, e.g., Ali et al., Mol. Cell. Proteomics. 2014, 13(12): 3396-3409, which is hereby incorporated by reference in its entirety.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice. The term “subject” as used herein refers to any member of the animal kingdom, such as a mammal. In one embodiment, the subject is a human. In another embodiment, the subject is a mouse.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. As used herein, and as well understood in the art, “to treat” or “treatment” is an approach for obtaining beneficial or desired results, including clinical results. Beneficial or desired clinical results can include, but are not limited to, alleviation or amelioration of one or more symptoms or signs, or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Treating also encompasses the prevention or amelioration of any symptom or symptoms or sign or signs of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

The term “a cell” includes a single cell as well as a plurality or population of cells. Administering a modulator or an agent to a cell includes both in vitro and in vivo administrations.

“Percentage of sequence identity” is determined by comparing two optimally aligned sequences over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more polypeptide sequences, refer to two or more sequences or subsequences that are the same or have a specified percentage of amino acid residues that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

“Percent (%) polypeptide sequence identity” with respect to polypeptide sequences is defined as the percentage of polypeptides in a candidate sequence that are identical with the polypeptides in the sequence of interest, after aligning the sequences and introducing gaps, if necessary, to achieve the maximum percent sequence identity. Alignment for purposes of determining % polypeptide sequence identity can be achieved in various ways that are within the skill in the art, for instance, using publicly available computer software such as BLAST, BLAST-2, or ALIGN software. Those skilled in the art can determine appropriate parameters for measuring alignment, including any algorithms needed to achieve maximal alignment over the full length of the sequences being compared.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. Proteins may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1 (2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The modulators, agents and/or pharmaceutical compositions described herein may be administered to, or used in, living organisms including humans, and animals. The term “subject” or “animal” as used herein refers to any member of the animal kingdom, in one embodiment a mammal such as a human being.

Administration of an “effective amount” of the modulators, agents and/or pharmaceutical compositions is defined as an amount effective, at dosages and for periods of time necessary to achieve the desired result. For example, an effective amount of a substance may vary according to factors such as the disease state, age, sex, and weight of the individual, and the ability of the recombinant protein to elicit a desired response in the individual. Dosage regime may be adjusted to provide the optimum therapeutic response. For example, several divided doses may be administered daily or the dose may be proportionally reduced as indicated by the exigencies of the therapeutic situation.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Expression of Lubricin in the Human Amniotic Membrane

Lubricin is the body's unique anti-adhesive, anti-fibrotic, anti-friction and anti-inflammatory glycoprotein (Schmidt T A et al., JAMA Ophthalmol 2013; 131:766-76 and Yu-Wai-Man C et al., JAMA Ophthalmol 2017; 135:1147-55). This amphiphile is a product of the proteoglycan 4 gene, contains a 1404-amino acid core, is extensively 0-glycosylated, and is characterized by a long, central mucin-like domain that permits lubricin to adhere and protect tissue surfaces. It is produced by numerous tissues, including the cornea, heart, lung, liver, cartilage, kidney, brain, testis, placenta and small intestine (Schmidt T A et al., JAMA Ophthalmol 2013; 131:766-76; Yu-Wai-Man C et al., JAMA Ophthalmol 2017; 135:1147-55; and genecards.org/cgi-bin/carddisp.pl?gene=PRG4, all of which are hereby incorporated by reference in their entireties). At these locations lubricin may regulate a number of processes, such as homeostasis, shear stress, tissue development, inflammation and wound healing (Schmidt T A et al., JAMA Ophthalmol 2013; 131:766-76 and Yu-Wai-Man C et al., JAMA Ophthalmol 2017; 135:1147-55).

The studies herein, were carried out to determine if lubricin is synthesized and expressed by the amniotic membrane (AM) and whether lubricin potentially plays a role, at least in part, in mediating anti-adhesive, anti-fibrotic and anti-inflammatory properties, and promotes tissue redevelopment and wound healing.

### Materials and Methods

Ten samples of human AMs and placentas were obtained as positive controls (Malhotra C, Jain A K. World J Transplant. 2014; 4:111-121) from the Tissue Repository of the Massachusetts General Hospital Pathology Service (Boston). These tissues originated from healthy donors (29-38 years old) following Caesarean sections and were de-identified before use. These AMs were also cryopreserved (CP) in glycerine (CP-AMs). The studies were approved by the Human Studies Committee of the Massachusetts Eye and Ear Infirmary (Boston). Two CP-AMs were also obtained (gift from Dr. Yukan Huang, Wuhan, China) and 6 freeze-dried AMs (PD-AM; Ruiji Bio-Engineering Co, Jiangxi, China). One of the FD-AMs was evaluated with or without incubation overnight at 4° C. with recombinant human (rh) lubricin (50 μL, 0.675 mg/mL; Lubris BioPharma, Framingham, Mass.). Samples were processed for immunofluorescence and Western blot analyses.

For histology, frozen sections (15 μM) were stained with hematoxylin and eosin or were incubated with an aliquot (1:50 dilution) of affinity-purified mouse antibody to human lubricin (Millipore Sigma, Burlington, Mass.) or the phosphate buffered saline, pH 7.4 (Boston BioProducts, Ashland, Mass.), diluent overnight at 4° C. The sections were then exposed to Donkey anti-mouse secondary antibody (1:500; Millipore) for 2 hours at room temperature and mounted using ProLong Gold antifade reagent with 4′,6-diamidino-2-phenylindole (DAPI; Invitrogen, Carlsbad, Calif.) for nuclear counterstaining. Slides were viewed with a Leica SP5 confocal microscope (Buffalo Grove, Ill.).

For protein determinations, Western blots were run, as reported (Schmidt T A, Sullivan D A, Knop E, et al. Transcription, translation, and function of lubricin, a boundary lubricant, at the ocular surface. JAMA Ophthalmol. 2013; 131:766-776, incorporated herein by reference in its entirety), using primary (1:1000) and secondary (1:5000) antibody incubation conditions as described previously. Recombinant human lubricin (rhlubricin) was included as a positive control.

### Results

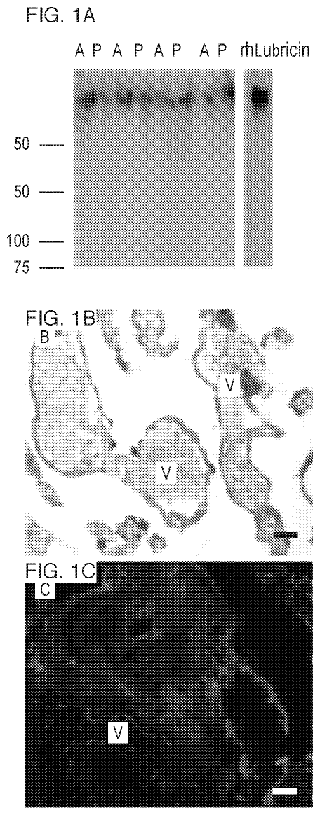

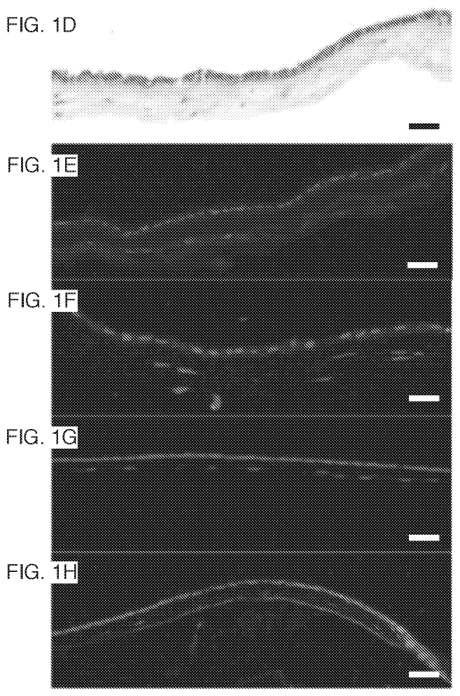

The results herein, demonstrate that all fresh placental (n=10) and fresh AM (n=10) samples contained lubricin (FIG. 1A). Lubricin was expressed in placental chorionic villi (FIGS. 1B, 1C), AM epithelial and stromal cells (FIGS. 1D, 1E), and CP-AM epithelia (FIG. 1F). All CP-AM samples (n=12) also contained lubricin, as shown by western blots. No lubricin could be detected in FD-AMs (n=6), either by immunofluorescence (FIG. 1G) or by western blots (n=7 experiments). Lubricin expression could be restored in FD-AMs after overnight incubation with rhlubricin (FIG. 1H).

The data described herein support the premise that lubricin is expressed in human AMs. In addition, the data show that preservation methods influence the extent of this expression. The earliest reported application of AM in ophthalmic surgery was in 1940 when it is used to reconstruct the ocular surface in patients with symblepharon (de Minh A, Arch Ophthalmol 1940; 23:3:522-525). Since that time, people have discovered the anti-adhesive, anti-fibrotic and anti-inflammatory features of AMs and used these tissues extensively to facilitate tissue reconstruction and wound healing (Malhotra C et al., World J Transplant 2014; 4:111-21). Because lubricin possesses innate anti-adhesive, anti-fibrotic, anti-friction and anti-inflammatory abilities, and is expressed by AMs, lubricin mediates biological properties of AMs.

Herein, lubricin was not detected in FD-AMs. The findings herein, provide evidence that the AM drying process leads to a disappearance of lubricin, which can be restored by lubricin exposure. The loss of lubricin may explain why dried, but not CP, AM loses its antiadhesive, antifibrotic, and antiinflammatory abilities that typically combine to inhibit scar formation. Indeed, this absence of lubricin may account for why the use of dried AM was unable to suppress inflammation, reduce adhesion generation, prevent scar formation, and decrease the risk of symblepharon development after strabismus surgery (Malhotra C, Jain A K. Human amniotic membrane transplantation: Different modalities of its use in ophthalmology. World J Transplant 2014; 4:111-121: A dR. Plastic repair of conjunctival defects with fetal membranes. Arch Ophthalmol 1940522-525; and Liu J, Sheha H, Fu Y, et al. Update on amniotic membrane transplantation. Expert Rev Ophthalmol 2010; 5:645-661, each of which is incorporated herein in the entirety).

How would the loss of lubricin in FD-AMs result in such sequelae? To explain, a scar is an area of fibrotic tissue that occurs after an injury and an adhesion is a band of scar tissue that binds 2 parts of tissue that are not normally joined together (eg, symblepharon). Scar and adhesion formations are often caused by fibrotic and inflammatory responses after tissue injury (Park D S J, et al. J Thorac Cardiovasc Surg. 2018; 156: 1598-1608.e1). Fibroblasts are found throughout the body and play an active role in producing the extracellular matrix (ECM). Fibroblasts also participate in the repair process by differentiating into myofibroblasts, which proliferate, migrate to the sites of injury, secrete cytokines, and promote the inflammatory response (Baum J, Duffy H S. J Cardiovasc Pharmacol. 2011; 57:376-379). This myofibroblast activation and associated ECM remodeling are important mechanisms by which mild adhesions transition to dense, fibrous adhesions. Proposed mechanisms to block adhesion formation include preventing myofibroblast proliferation and reducing the initial inflammatory response (Oh J, et al. J. Surg Res. 2017; 208:20-25).

Lubricin possesses innate antifibrotic and antiinflammatory properties that retard adhesion development after tissue injury. For example, lubricin treatment suppresses myofibroblast proliferation and ECM remodeling, decreases fibrotic and inflammatory responses, and/or prevents adhesion formation in the pericardial and abdominal cavities, lens, tendons, and joints. Lubricin acts directly on fibroblasts and immune cells by binding to CD44, toll like receptor (TLR)2, and TLR4 receptors and suppressing IκBα phosphorylation and NFκB translocation. Lubricin also significantly reduces the interleukin (IL)-1β-induced increase in IL-6, IL-8, and COX2 expression, as well as that of matrix metalloproteinases-1, -3, -9, and -13, which are involved in fibroblast proliferation and migration (Bartok B, Firestein G S. Immunol Rev. 2010; 233:233-255; Xue M, McKelvey K, Shen K, et al. Endogenous MMP-9 and not MMP-2 promotes rheumatoid synovial fibroblast survival, inflammation and cartilage degradation. Rheumatology (Oxford). 2014; 53:2270-2279).

Of particular interest, lubricin may have other applications related to the eye. The inventors have discovered in a clinical trial that topical rhlubricin significantly reduces the signs and symptoms of dry eye disease (Lambiase A, Sullivan B D, Schmidt T A, et al. A two-week, randomized, double-masked study to evaluate safety and efficacy of lubricin (150 μg/mL) eye drops versus sodium hyaluronate (HA) 0.18% eye drops (vismed (R)) in patients with moderate dry eye disease. Ocul Surf. 2017; 15:77-87, incorporated herein by reference in its entirety). This disease is characterized by a vicious cycle of tear film hyperosmolarity and instability and leads to increased friction, inflammation, eye damage, pain, and visual impairment (Bron, A J et al., Ocul Surf. 2017; 15:438-510). Currently, there is no global cure for dry eye. In addition, others have found that a down regulation of the lubricin gene in human conjunctival fibroblasts correlates with multiple failed glaucoma operations and worse visual acuities in patients (Yu-Wai-Man C. et al. JAMA Ophthalmol. 2017; 135:1147-1155). Furthermore, binding of lubricin to contact lenses may decrease their friction and improve their comfort.

As one additional consideration, the ability of lubricin to prevent adhesion formation could have yet multiple other clinical applications for the eye. Topical lubricin could possibly prevent the development of symblephara that are known to occur in numerous pathological conditions, including pterygia, ocular cicatricial pemphigoid, Stevens-Johnson syndrome, erythema multiforme, conjunctivitis (ie, bacterial, viral, vernal, and atopic), porphyria cutanea tarda, rosacea, xeroderma pigmentosum, and squamous papilloma of the conjunctiva. If so, this might remove the necessity of surgical approaches, such as AM transplants, to treat these adhesions. Of particular interest, preservation appears to reduce or remove lubricin in CP-AM or FD-AM, respectively, but this expression could be restored by lubricin exposure. Alternatively, lubricin alone may be used as a treatment in various pathological conditions.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

While this invention has been particularly shown and described with references to preferred embodiments thereof, it will be understood by those skilled in the art that various changes in form and details may be made therein without departing from the scope of the invention encompassed by the appended claims.

### INCORPORATION BY REFERENCE

The patent and scientific literature referred to herein establishes the knowledge that is available to those with skill in the art. All United States patents, published or unpublished United States patent applications, and PCT patent applications designating the U.S. cited herein are incorporated by reference in their entirety. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed.

1. A pharmaceutical composition for treating tissue reconstruction and wound healing in a subject, comprising a therapeutically effective amount of a lubricin-supplemented preserved amniotic membrane (AM) or a lubricin-supplemented non-AM substrate.

2. The pharmaceutical composition of claim 1, wherein said preserved AM comprises a cryopreserved AM (CP-AM) or a freeze dried AM (FD-AM).

3. The pharmaceutical composition of claim 1, wherein said lubricin-supplemented preserved AM is a FD-AM soaked in, rehydrated with, or incubated with lubricin.

4. The pharmaceutical composition of claim 1, wherein said lubricin-supplemented non-AM substrate is a non-AM substrate soaked in, rehydrated with, or incubated with lubricin.

5. The pharmaceutical composition of claim 1, wherein said pharmaceutical composition promotes tissue reconstruction, prevents adhesion formation, alleviates obstructions, facilitates wound healing or performs any combinations thereof.

6. The pharmaceutical composition of claim 5, wherein said pharmaceutical composition promotes tissue reconstruction of an ocular surface, an oral surface, a periodontal surface, an abdominal surface, a vaginal surface, a cervical surface, a uterine surface, a skin surface or a mucosal surface.

7. The pharmaceutical composition of claim 5, wherein said pharmaceutical composition prevents adhesion formation of an ocular surface, nasolacrimal duct, intrauterine, fallopian tube, or post-radiation tissue damage.

8. The pharmaceutical composition of claim 5, wherein said pharmaceutical composition alleviates obstructions of nasolacrimal duct or fallopian tube.

9. The pharmaceutical composition of claim 5, wherein said pharmaceutical composition facilitates wound healing of a burn injury, an epithelial defect or an ulcer.

10. The pharmaceutical composition of claim 5, wherein said pharmaceutical composition facilitates wound healing or tissue reconstruction of an ocular surface disease.

11. The pharmaceutical composition of claim 9, wherein said ocular surface disease comprises a sign of conjunctival congestion, conjunctival/cornea ulceration, corneal edema, corneal clouding, extensive fluorescence staining, neovascularization, aqueous flare, aqueous cells in anterior chamber, corneal perforation, corneal/conjunctival epithelial demarcation, corneal stromal inflammation, corneal thinning, cornea stromal edema, corneal endothelial inflammatory plaque, Descemet's folds, conjunctival mucopurulent discharge, anterior chamber reaction and hypopyon, upper eyelid edema, posterior synechiae, hyphema, high intraocular pressure, loss of ocular contents, iris prolapse, or severe dry eye.

12. The pharmaceutical composition of claim 1, wherein said pharmaceutical composition reduces or relieves a symptom or a sign of dacryocystitis.

13. A method of treating tissue reconstruction or wound healing in a subject, comprising contacting a wound or surgical site with a composition comprising a therapeutically effective amount of a lubricin-supplemented preserved AM or a lubricin-supplemented non-AM substrate.

14. The method of claim 13, wherein said preserved AM comprises a cryopreserved AM (CP-AM) or a freeze dried AM (FD-AM).

15. The method of claim 13, wherein said lubricin-supplemented preserved AM is a FD-AM soaked in, rehydrated with or incubated with lubricin.

16. The method of claim 13, wherein said lubricin-supplemented non-AM substrate is a non-AM substrate soaked in, rehydrated with or incubated with lubricin.

17. The method of claim 13, wherein said composition promotes tissue reconstruction, prevents adhesion formation, alleviates obstructions, facilitates wound healing or performs any combinations thereof.

18. The method of claim 17, wherein said composition promotes tissue reconstruction of an ocular surface, an oral surface, a periodontal surface, an abdominal surface, a vaginal surface, a cervical surface, a uterine surface, a skin surface, or a mucosal surface.

19. The method of claim 17, wherein said composition prevents adhesion formation of an ocular surface, nasolacrimal duct, intrauterine, fallopian tube, or post-radiation tissue damage.

20. The method of claim 17, wherein said composition alleviates obstructions of nasolacrimal duct or fallopian tube.

21. The method of claim 17, wherein said composition facilitates wound healing of a burn injury, an epithelial defect or an ulcer.

22. The method of claim 13, wherein said composition reduces or relieves a symptom or a sign of a fallopian tube abnormality, an intrauterine adhesion or associated female infertility.

23. The method of claim 22, wherein said symptom or sign is associated with a pelvic inflammatory disease, a pathogen infection, endometriosis, an adhesion from previous surgery, an adhesion from nontubal infection, pelvic tuberculosis, salpingitis isthmica nodosa, a plug of mucus and amorphous debris, a spasm of a uterotubal ostium, a hydrosalpinx or any combinations thereof.

24. The method of claim 17, wherein said composition facilitates wound healing or tissue reconstruction of an ocular surface disease.

25. The method of claim 24, wherein said ocular surface disease comprises a sign of conjunctival congestion, conjunctival/cornea ulceration, corneal edema, corneal clouding, extensive fluorescence staining, neovascularization, aqueous flare, aqueous cells in anterior chamber, corneal perforation, corneal/conjunctival epithelial demarcation, corneal stromal inflammation, corneal thinning, cornea stromal edema, corneal endothelial inflammatory plaque, Descemet's folds, conjunctival mucopurulent discharge, anterior chamber reaction and hypopyon, upper eyelid edema, posterior synechiae, hyphema, high intraocular pressure, loss of ocular contents, iris prolapse, or severe dry eye.

26. The method of claim 13, wherein said composition reduces or relieves a symptom or a sign of dacryocystitis.

27.-32. (canceled)

33. A method of preparing a lubricin-supplemented preserved amniotic membrane (AM) or a lubricin-supplemented non-AM substrate, comprising soaking, rehydrating or incubating a preserved AM or a non-AM substrate with lubricin.

34. The method of claim 33, wherein said preserved AM comprises a cryopreserved AM (CP-AM) or a freeze dried AM (FD-AM).

35. The method of claim 34, wherein said preserved AM comprises a FD-AM.

36.-37. (canceled)
